(19) United States
(12) Patent Application Publication
(10) Pub. No.: US 2024/0285489 A1
(43) Pub. Date: Aug. 29, 2024

(54) AQUEOUS ANTIPERSPIRANT COMPOSITIONS

(71) Applicant: Conopco, Inc., d/b/a UNILEVER, Englewood Cliffs, NJ (US)

(72) Inventors: Cameron Kay, York (GB); Craig James Luckwell, Leeds (GB)

(21) Appl. No.: 18/572,017

(22) PCT Filed: Jul. 4, 2022
(86) PCT No.: PCT/EP2022/068454

§ 371 Date: Dec. 19, 2023

(30) Foreign Application Priority Data
Jul. 7, 2021 (EP) 21184313.1

(51) Int. Cl. A61K 8/30; A61Q 15/00

(52) U.S. Cl. CPC A61K 8/30; A61Q 15/00

(57) ABSTRACT

An aqueous composition comprising an aluminium-zirconium-glycine complex which is an aluminium-zirconium tri-, tetra-, or penta-chlorohydrex-glycine complex and a water-soluble calcium salt, the molar ratio of aluminium to calcium being from 1.3 to 60 and the molar ratio of aluminium to glycine being from 1.5 to 25.

### FIELD OF INVENTION

The invention is concerned with aqueous antiperspirant (AP) composition, particularly such compositions having enhanced activity.

### BACKGROUND

Aluminium-containing AP salts having enhanced activity are well known in the prior art. The AP salts produced are often described as “activated”.

Traditionally, activated AP salts have been prepared by prolonged heating of basic aluminium chloride solutions followed by spray-drying; see, for example, U.S. Pat. No. 4,359,456 (Gosling). The samples prepared by this method needed to be formulated into essentially anhydrous compositions in order for the AP salt to maintain its high activity.

Activated AP salts have also been prepared using water-soluble calcium acids, particularly with a further adjunct such as an amino acid, hydroxyl acid, or betaine. Some of these samples could be formulated into aqueous compositions without the AP salt losing all of its enhanced activity.

EP 1,104,282 (Gillette) discloses a means of producing activated AP salts using a water-soluble calcium salt and glycine or a hydroxy acid.

U.S. Pat. No. 6,911,195 (Gillette) discloses water-in-oil emulsion gels comprising aluminium-zirconium AP salts activated using calcium ions.

U.S. Pat. No. 5,955,065 (Gillette) discloses anhydrous suspension formulations comprising particulate AP salts and aluminium-zirconium AP salts activated using calcium ions.

U.S. Pat. No. 6,942,850 (Gillette) discloses aqueous alcoholic composition comprising aluminium-zirconium AP salts activated using calcium ions.

WO 2009/044381 (P&G) discloses water-in-oil emulsion sticks comprising aluminium and aluminium-zirconium AP salts activated using calcium ions.

U.S. Pat. No. 7,704,531 (Colgate) discloses compositions comprising an active system made from combining an aluminium or aluminium-zirconium salt, a calcium salt, and a betaine.

US 2011/0038823 (Dial/Henkel) discloses water-in-oil emulsion sticks comprising an AP active prepared by combining BAC, calcium chloride and glycine.

US 2007/196303, US 2007/0020211, WO 2008/063188, US 2008/0131354 and U.S. Pat. No. 7,087,220 (Summit and Reheis) each describe methods of making calcium-activated AP salts.

US 2007/0003499 (P&G) discloses enhanced efficacy aluminium-zirconium chlorohydrex acid complexes in combination with a neutralizing salt, the AP salt having a pH at 15% by weight of greater than 5.

### SUMMARY OF INVENTION

The invention is concerned with aqueous AP compositions comprising AP complexes of enhanced activity and their method of manufacture. The AP complexes involved are specific aluminium-zirconium complexes with glycine.

It is an object of the invention to provide aqueous AP compositions having superior efficacy.

It is a further object of the invention to provide aqueous AP compositions that have low irritation potential.

It is a further object of the invention to provide aqueous AP compositions that have good rheological stability.

In a first aspect of the invention, there is provided an aqueous composition comprising an aluminium-zirconium-glycine complex which is an aluminium-zirconium tri-, tetra-, or penta-chlorohydrex-glycine complex and a water-soluble calcium salt, the molar ratio of aluminium to calcium being from 1.3 to 60 and the molar ratio of aluminium to glycine being from 1.5 to 25.

Aqueous compositions according to the first aspect of the invention are typically AP compositions, i.e. compositions that reduce perspiration on topical application. In some alternative embodiments, aqueous compositions according to the first aspect of the invention may be used in the manufacture of AP compositions.

In a second aspect of the invention, there is provided a method of manufacture of an AP composition according to the first aspect of the invention.

In a third aspect of the invention, there is provided a cosmetic method of obtaining an antiperspirancy benefit comprising the topical application to the surface of the human body of an AP composition according to the first aspect of the invention.

### DETAILED DESCRIPTION

Herein, features expressed as “preferred” with regard to a particular aspect of the invention should be understood to be preferred with regard to each aspect of the invention (likewise, features expressed as “more preferred”, “particularly preferred” or “most preferred”). “Preferred” features aid the delivery of one or more of the objects of the invention.

Herein, preferred features of the invention are particularly preferred when used in combination with other preferred features (likewise, features expressed as “more preferred”, “particularly preferred” or “most preferred”).

Herein, “ambient conditions” refer to 20° C. and 1 atmosphere pressure, unless otherwise indicated.

Herein, all percentages, ratios and amounts are by weight, unless otherwise indicated. Herein, amounts and concentrations of ingredients are percentages by weight of the total composition, unless otherwise indicated.

Herein, ratios expressed as, for example, “Al:Ca is from 1.3 to 60”, means that Al:Ca is from 1.3:1 to 60:1.

Herein, references to molar amounts and ratios of “aluminium” are calculated on the basis of mono-nuclear aluminium, but include aluminium present in poly-nuclear species; indeed, most of the aluminium in the salts of relevance is present in poly-nuclear species.

Herein, references to amounts of components such as “carrier oil” or “thickening agent” relate to the total amount of such components present in the composition.

Herein, the word “comprising” is intended to mean “including” but not necessarily “consisting of”, i.e., it is non-exhaustive.

Herein, “cosmetic” methods and compositions should be understood to mean non-therapeutic methods and compositions, respectively.

Herein the term “AP salt” is sometimes used to refer to the aluminium-zirconium-glycine complex used in the invention.

The aqueous AP compositions of the invention deliver surprisingly good antiperspirancy performance. In addition, such compositions have good storage stability, in particular rheological stability. Further, such compositions have low irritancy due to their relatively high pH.

The AP salt used in the invention is typically manufactured from an aluminium-zirconium tri- tetra- or penta-chlorohydrex-glycine complex through “activation” with a water-soluble calcium salt (vide infra).

Herein, AP salts referred to as “octa salts”, including those complexed with glycine, are aluminium-zirconium octa-chlorohydrex species wherein the aluminium to zirconium ratio is from 6 to 10 and the metal to chloride ratio is from 0.9 to 1.5. That is to say:

$$\begin{matrix}Al : Zr & = & 6 : 1 to 10 : 1 \\ and ( Al + Zr ) : Cl & = & 0.9 : 1 to 1.5 : 1.\end{matrix}$$

An example of such a salt is Al₈Zr(OH)₂₀Cl₈, wherein Al:Zr is 8 and (Al+Zr):Cl is 1.125.

Herein, AP salts referred to as “penta salts”, including those complexed with glycine, are aluminium-zirconium penta-chlorohydrex species wherein the aluminium to zirconium ratio is from 6 to 10 and the metal to chloride ratio is from 1.51 to 2.1. That is to say:

$$\begin{matrix}Al : Zr & = & 6 : 1 to 10 : 1 \\ and ( Al + Zr ) : Cl & = & 1.51 : 1 to 2.1 : 1.\end{matrix}$$

An example of such a salt is Al₈Zr(OH)₂₃Cl₅, wherein Al:Zr is 8 and (Al+Zr):Cl is 1.8.

Penta salts used in accordance with the invention are complexed with glycine.

Herein, AP salts referred to as “tetra salts”, including those complexed with glycine, are aluminium-zirconium tetra-chlorohydrex species wherein the aluminium to zirconium ratio is from 2 to 5.99 and the metal to chloride ratio is from 0.9 to 1.5. That is to say:

$$\begin{matrix}Al : Zr & = & 2 : 1 to 5.99 : 1 \\ and ( Al + Zr ) : Cl & = & 0.9 : 1 to 1.5 : 1.\end{matrix}$$

An example of such a salt is Al₄Zr(OH)₁₂Cl₄, wherein Al:Zr is 4 and (Al+Zr):Cl is 1.25.

Tetra salts used in accordance with the invention are complexed with glycine.

Herein, AP salts referred to as “tri salts”, including those complex with glycine, are aluminium-zirconium tri-chlorohydrex species wherein the aluminium to zirconium ratio is from 2 to 5.99 and the metal to chloride ratio is from 1.51 to 2.1. That is to say:

$$\begin{matrix}Al : Zr & = & 2 : 1 to 5.99 : 1 \\ and ( Al + Zr ) : Cl & = & 1.51 : 1 to 2 : 1.\end{matrix}$$

An example of such a salt is Al₄Zr(OH)₁₃Cl₃, wherein Al:Zr is 4 and (Al+Zr):Cl is 1.667.

Tri salts used in accordance with the invention are complexed with glycine.

The AP used in the invention is preferably an aluminium-zirconium tetra- or penta-chlorohydrex-glycine complex and is more preferably an aluminium-zirconium penta-chlorohydrex-glycine complex. Even more preferably it is a calcium-activated aluminium-zirconium penta-chlorohydrex-glycine complex.

In compositions according to the invention, the concentration of the aluminium-zirconium tri- tetra- or penta-chlorohydrex-glycine complex is preferably from 20 to 60%, more preferably from 25 to 50% and most preferred from 30 to 45%, including all of the glycine present, whether present in an Al—Zr complex added or independently added. When compositions according to the invention are used in manufacture of an AP composition, the above concentrations are particularly relevant.

Preferred water-soluble calcium salts used in accordance with the invention have a solubility in water of 10 g/L or greater, e.g. 10-100 g/L, under ambient conditions. A particularly preferred water-soluble calcium salt is calcium chloride.

The content of water-soluble calcium salt in compositions of the invention is preferably from 3 to 20%, more preferably from 4 to 15% and most preferred from 6 to 12.5%.

The level of water-soluble calcium salt used in the invention is such that the molar ratio of aluminium to calcium is from 1.3 to 60 and preferably from 1.8 to 20. For compositions comprising aluminium-zirconium penta-chlorohydrex-glycine complexes, the molar ratio of aluminium to calcium is preferably from 1.5 to 60, more preferably from 1.8 to 20, still more preferably from 2 to 10 and most preferably from 2 to 7.

The glycine used in the invention may be used per se or as a salt thereof, such glycine hydrochloride. It may also be used as a pre-formed complex with an aluminium-zirconium tri- tetra- or penta-chlorohydrex salt. Amounts and ratios relating to glycine are to the total glycine content per se.

The total content of glycine in compositions of the invention is preferably from 1 to 15% and more preferably from 2 to 12%. For compositions comprising aluminium-zirconium penta-chlorohydrex-glycine complexes, the total content of glycine is preferably from 1 to 15%, more preferably from 2 to 12% and most preferably from 3 to 10%.

The total level of glycine used is such that the molar ratio of aluminium to glycine is from 1 to 25 and preferably from 1.5 to 15. For compositions comprising aluminium-zirconium penta-chlorohydrex-glycine complexes, the molar ratio of aluminium to glycine is preferably from 1.5 to 20, more preferably from 2 to 10 and most preferably from 2 to 6.

Compositions of the invention comprise a mixture of aluminium-zirconium species having a relatively high content of what is commonly termed Band 3 material, as determined by SEC (Size Exclusion Chromatography). The SEC technique employed is well known in the art and is described in further detail in U.S. Pat. No. 4,359,456 (Gosling). The SEC band commonly referred to as Band 3 is designated as “Peak 4” in EP 1,104,282 B1 by Gillette. Having a high content of Band 3 material has been associated with high antiperspirancy performance in the prior art.

Herein, “Band 3 content” refers to the integrated area in the Band 3 region of the SEC chromatograph relative to the total integrated area in all of the regions corresponding to aluminium species; that is to say, Bands 1, 2, 3, and 4.

The Band 3 content of compositions produced according to the invention is preferably at least 30%, more preferably at least 50% and most preferably at least 70%.

In the method of manufacture described herein, it is preferred that the mixture is heated for sufficient time and at sufficient temperature for the Band 3 content of the aluminium-zirconium species to become at least 30%, more preferably at least 50% and most preferably at least 70%.

The Band 3:Band 2 ratio is a further criterion by which the extent of activation of the Al/Zr AP salt may be judged. The Band 3:Band 2 ratio is the integrated area in the Band 3 region of the SEC chromatograph divided by the integrated area in the Band 2 region. The Band 3 to Band 2 ratio in the actives resulting from the manufacturing method of the invention is preferable at least 3, for example from 3 to 10, and more preferably at least 5, for example from 5 to 10.

The process used to manufacture compositions of the invention typically involves the heating together in aqueous solution of an aluminium-zirconium tri- tetra- or penta-chlorohydrex-glycine complex and a water-soluble calcium salt. In some embodiments, the aluminium-zirconium tri- tetra- or penta-chlorohydrex-glycine complex is produced in situ from an aluminium-zirconium tri- tetra- or penta-chlorohydrex salt and glycine, by heating in aqueous solution with the water-soluble calcium salt.

The heating together referred to in the paragraph immediately above causes “activation” of the antiperspirant salt, increasing its Band 3 content and its Band 3:Band 2 ratio (vide infra). This higher Band 3 content leads to a more effective antiperspirant active, as previously recognised in the literature.

In preferred embodiments, the method of manufacture involves heating the components together for 30 minutes or longer at a temperature of 50° C. or above. In particularly preferred embodiments, the method involves heating the components together for 60 minutes or longer at a temperature of 70° C. or above, for example 1 to 2 hours at 70° C. to 90° C.

Following this heat activation step, the aqueous solution is typically allowed to cool to ambient temperature. The solution may then be used in the immediate preparation of antiperspirant compositions or it may be stored before being used. Aqueous AP salt solutions according to the invention have been found to have good storage stability, particularly the tetra- and penta salts (vide infra).

The aqueous AP salt solution resulting from the activation step may be used “as is” or it may be diluted or concentrated prior to use. One form of “concentration” involves spray drying of the aqueous antiperspirant salt solution to give a particulate solid. This is typically milled and may be used in a range of AP compositions, such as suspension sticks and soft solids.

The pH of the aqueous compositions of the invention is preferably from 3.5 to 4.1 and more preferably from 3.6 to 4.0. These are relatively high pH ranges for AP solutions and can assist with reducing potential irritation.

A preferred additional component of compositions of the invention is a C2-C4 alcohol or polyol, such as ethanol, propylene glycol or glycerol. Ethanol is particularly preferred. The C2-C4 alcohol or polyol, or ethanol, is incorporated at a preferred level of from 2 to 80%, more preferably from 5 to 70% and most preferably from 15 to 60% by weight of the total composition.

A preferred additional component of compositions of the invention is an oil.

Herein, the terms “oil” and signifies a water-insoluble organic material that is liquid at 20° C. Any material having a solubility of less than 0.1 g/100 g at 20° C. is considered to be insoluble.

A preferred oil for use in compositions in accordance with the invention is a fragrance oil, sometimes alternatively called a perfume oil. The fragrance oil may comprise a single fragrance or component more commonly a plurality of fragrance components. Herein, fragrance oils impart an odour, preferably a pleasant odour, to the composition. Preferably, the fragrance oil imparts a pleasant odour to the surface of the human body the composition is applied to the same.

The amount of fragrance oil in the composition is commonly up to 3% advantageously is at least 0.5% and particularly from 0.8% to 2%.

The total amount of oil in the composition is preferably from 0.1 to 20%, more preferably from 0.5 to 10%, and most preferably at from 2 to 8% by weight of the total composition. In certain preferred embodiments, particularly those also comprising an aluminium and/or zirconium containing AP active, the oil is present at greater than 2.5% and less than 6% by weight of the total composition.

In certain embodiments, it is preferred to include an oil, other than a fragrance oil, that has a relatively low viscosity, by which is meant less 250 cS (mm²·s⁻¹). Such oils can improve the sensory properties of the composition on application and can lead to other benefits such as emolliency.

Suitable oils can be selected from alkyl ether oils having a boiling point of above 100° C. and especially above 150° C., including polyalkyleneglycol alkyl ethers. Such ethers desirably comprise between 10 and 20 ethylene glycol or propylene glycol units and the alkyl group commonly contains from 4 to 20 carbon atoms. The preferred ether oils include polypropylene glycol alkyl ethers such as PPG-14-butylether and PPG-15-stearyl ether.

Suitable oils can include one or more triglyceride oils. The triglyceride oils commonly comprise the alkyl residues of aliphatic C7 to C20 alcohols, the total number of carbon atoms being selected in conjunction with the extent of olefinic unsaturation and/or branching to enable the triglyceride to be liquid at 20° C. One example is jojoba oil. Particularly preferably, in the triglyceride oil the alkyl residues are linear C₁₈ groups having one, two or three olefinic degrees of unsaturation, two or three being optionally conjugated, many of which are extractable from plants (or their synthetic analogues), including triglycerides of oleic acid, linoleic acid, conjugated linoleic acids, linolenic acid, petroselenic acid, ricinoleic acid, linolenelaidic acid, trans 7-octadecenoic acid, parinaric acid, pinolenic acid, punicic acid, petroselenic acid and stearidonic acid.

Suitable oils can include those derived from unsaturated C₁₈ acids, including coriander seed oil, impatiens balsimina seed oil, parinarium laurinarium kernel fat oil, sabastiana brasilinensis seed oil, dehydrated castor seed oil, borage seed oil, evening primrose oil, aquilegia vulgaris oil, sunflower (seed) oil and safflower oil. Other suitable oils are obtainable from hemp, and maize corn oil. An especially preferred oil by virtue of its characteristics is sunflower (seed) oil.

Further suitable oils, that can also be emollient oils, comprise alkyl or alkyl-aryl ester oils having a boiling point of above 150° C. (and a melting point of below 20° C.). Such ester oils include oils containing one or two alkyl groups of 12 to 24 carbon atoms length, including isopropyl myristate, isopropyl palmitate and myristyl palmitate. Other non-volatile ester oils include alkyl or aryl benzoates such C₁₂₋₁₅ alkyl benzoate, for example Finsolv TN™ or Finsolv Sun™.

Preferred compositions of the invention comprise an emollient oil or a fragrance.

A further class of suitable oils comprises non-volatile dimethicones, often comprising phenyl or diphenylene substitution, for example Dow Corning 200 350 cps or Dow Corning 556.

In compositions of the invention comprising an oil, an emulsifier is a preferred additional component. In some preferred embodiments, particular those comprising an oil-in-water emulsion, it is preferred that emulsifier(s) form a lamellar phase emulsifier system in the composition. Such systems lead to good emulsion stability in compositions according to the invention.

It is preferred that compositions of the invention comprise a non-ionic emulsifier system. Such an emulsifier system preferably has a mean HLB value in the region of from about 5 to about 12 and particularly from 6 to about 10. An especially desired mean HLB value is from 6 to 9. Such a mean HLB value can be provided by selecting an emulsifier having such an HLB value, or more preferably by employing a combination of at least two emulsifiers, a first (lower) HLB emulsifier having an HLB value in the range of from 2 to 6.5, such as in particular from 4 to 6 and a second (higher) HLB emulsifier having an HLB value in the range of from about 6.5 to 18 and especially from about 12 to about 18. When a combination of emulsifiers is employed, the average HLB value can be calculated as a weight average of the HLB values of the constituent emulsifiers.

An especially desirable range of emulsifiers comprises a hydrophilic moiety provided by a polyalkylene oxide (polyglycol), and a hydrophobic moiety provided by an aliphatic hydrocarbon, preferably containing at least 10 carbons and commonly linear. The hydrophobic and hydrophilic moieties can be linked via an ester or ether linkage, possibly via an intermediate polyol such as glycerol. A preferred range of emulsifiers comprises polyethylene glycol ethers.

The total proportion of emulsifiers in the composition is preferably at least 0.5% and particularly at least 1% by weight. Commonly, the emulsifiers are not present at above 10%, often not more than 7% by weight and in many preferred embodiments up to 6% by weight. An especially desirable concentration range for the emulsifiers is from 2.5 to 5% by weight.

A further optional component of compositions of the invention is a thickening agent, sometimes alternatively referred to as a gelling agent or gellant.

The thickening agent may be selected from any of those known in the art. Often, the thickening agent includes a wax. Waxes typically are considered to melt at above 40° C. and particularly between 55 and 95° C. Waxes can include ester waxes, including C12 to C24 linear fatty alcohols, waxes obtained from animals or plants, often after hydrogenation, silicone elastomers and silicone waxes. The thickening agent can comprise a mixture of particulate thickening agents, a mixture of waxes or a mixture of both types of material.

Other thickening agents that may be used include cellulose derivatives, such as hydroxyethyl cellulose and hydroxypropyl cellulose. These thickening agents are particularly suitable for liquid compositions, such as roll-on compositions.

Thickening agents may be used at from 0.5 to 30%, with levels such as from 10 to 20% preferred in solid compositions, such as emulsion stick compositions.

Other components that may be included in compositions according to the invention including those described in the following paragraphs.

Wash-off agents may be included, often in an amount of up to 10%, to assist in the removal of the formulation from skin or clothing. Such wash-off agents are typically non-ionic surfactants such as esters or ethers containing a C to C₂₂ alkyl moiety and a hydrophilic moiety comprising a polyoxyalkylene group (POE or POP).

Skin feel improvers (e.g. talc or finely divided high molecular weight polyethylene), may be included, typically in an amount from 1 up to 10%.

Skin moisturisers, such as glycerol or polyethylene glycol (e.g. mol. wt. 200 to 600) may be included, typically in an amount of up to 5%.

Skin benefit agents, such as allantoin or lipids, may be included, typically in an amount of up to 5%.

An optional component is a preservative, such as ethyl or methyl parabens or BHT (butyl hydroxy toluene), typically in an amount of from 0.01 to 0.1%.

### EXAMPLES

The commercially available Al—Zr AP actives indicated in Table 1 were used to prepare the following Examples and Comparative Examples. Ratios provided in the supplier's certificates of analysis were used to calculate average molecular weights and hence estimate the molar amounts used in their manufacture, as indicated in Table 1. Each active was supplied and used as an aqueous solution with the indicated levels of anhydrous solids.

TABLE 1

| | Av. Mol. Wt. | Al:Zr | Metal:CI | Glycine | Anh. solids |
|---|---|---|---|---|---|
| AP Salt | (g · mol⁻¹) | Ratio | Ratio | (% w/w) | (% w/w) |
| Octa | 948.8 | 8.41 | 1.30 | 3.3 | 32.0 |
| Penta | 761.7 | 6.70 | 1.65 | 0.0 | 36.4 |
| Tetra | 494.4 | 3.54 | 1.42 | 4.3 | 27.8 |
| Tri | 680.2 | 5.69 | 1.60 | 4.6 | 28.0 |

The AP solutions indicated in Table 1 were used to prepare activated Al—Zr AP species according to Tables 2 to 5, using the following procedure.

Into 50 ml polypropylene tubes were added: deionised water (0 to 4 g, 0 to 222 mmol) and AP solution (14 to 19 g), followed by calcium chloride dihydrate (1 to 3 g, 6.8 to 20.4 mmol) and glycine as required. The weights of the components always totaled 20 g. The tubes were then capped before being shaken to observe full dissolution of the solids. The tubes were placed in racks (8 per rack), which were each placed in a water bath and heated to 87° C. The tubes were left for 2 hours at 87° C., with shaking of each tube individually at 10-minute intervals. After the two hours the racks were removed from the water bath and allowed to cool to room temperature.

HPLC analysis of each of the species produced was performed by the following method and the content of “Band 3” material is indicated in the final column of Tables 2-5 (average values and standard errors indicated are based on 3 measurements).

SEC-HPLC chromatograms were recorded using a Dionex Ultimate 3000 HPLC system and Shodex RI-101 detector using two 5 μm silica porous columns in series and the chromeleon software. The program was set to inject 20 μl of sample and record the refractive index (RI) detector response over 10 minutes. The mobile phase of the column was a mixture of 0.01 M HNO₃ and 0.1 M NaNO₃ at a flow rate of 0.75 ml·min⁻¹. Liquid samples were made up using 0.5 g of liquid and diluted to 10 g with 0.01 M HNO₃. Solid samples were made up using 0.2 g of solid and diluted up to 10 g with 0.01 M HNO₃. Data were recorded 24 hours after the solutions were manufactured and at 25° C.

TABLE 2
Octa Salts

| | | | Additional | Additional | |
|---|---|---|---|---|---|
| | AP soln. | CaCl₂ | Glycine | Water | Band 3 |
| Sample ID | (% w/w) | (% w/w) | (% w/w) | (% w/w) | HPLC (%) |
| Octa 1 | 83.2 | 7.8 | 2.4 | 6.6 | (40.4 ± 0.6) |
| Octa 2 | 87.0 | 8.1 | 2.0 | 2.9 | (39.2 ± 0.8) |
| Octa 3 | 78.2 | 7.3 | 1.9 | 12.6 | (45.1 ± 0.2) |
| Octa 4 | 75.7 | 7.5 | 1.9 | 14.9 | (43.5 ± 0.5) |
| Octa 5 | 77.0 | 7.1 | 2.0 | 13.9 | (42.4 ± 0.9) |
| Octa 6 | 85.6 | 6.5 | 1.9 | 6.0 | (32.6 ± 0.3) |
| Octa 7 | 80.0 | 10.1 | 1.9 | 8.0 | (42.5 ± 0.6) |
| Octa 8 | 71.6 | 9.5 | 1.9 | 17.0 | (50.1 ± 0.5) |
| Octa 9 | 85.0 | 11.6 | 2.1 | 1.7 | (43.0 ± 0.3) |
| Octa 10 | 90.0 | 5.0 | 2.0 | 2.9 | (39.1 ± 0.8) |
| Octa 11 | 85.2 | 8.9 | 2.5 | 3.4 | (46.4 ± 0.8) |
| Octa 12 | 78.1 | 8.4 | 1.5 | 12.0 | (40.8 ± 0.2) |
| Octa 13 | 78.1 | 9.1 | 2.0 | 10.3 | (52.3 ± 0.7) |
| Octa 14 | 83.0 | 8.9 | 2.1 | 6.1 | (43.9 ± 0.7) |

TABLE 3
Tri Salts

| | | | Additional | Additional | Band 3 |
|---|---|---|---|---|---|
| | AP soln. | CaCl₂ | Glycine | Water | HPLC |
| Sample ID | (% w/w) | (% w/w) | (% w/w) | (% w/w) | (%) |
| Tri 1 | 75.0 | 7.4 | 2.4 | 15.2 | (63.6 ± 0.6) |
| Tri 2 | 84.4 | 9.3 | 2.2 | 4.1 | (59.0 ± 0.9) |
| Tri 3 | 82.4 | 9.4 | 1.8 | 6.4 | (57.5 ± 0.5) |
| Tri 4 | 72.0 | 8.1 | 1.7 | 18.2 | (59.8 ± 0.6) |
| Tri 5 | 88.4 | 5.7 | 2.2 | 3.7 | (50.6 ± 0.3) |
| Tri 6 | 76.3 | 12.0 | 2.6 | 9.1 | (60.1 ± 0.8) |
| Tri 7 | 78.5 | 9.6 | 1.2 | 10.6 | (52.0 ± 0.9) |
| Tri 8 | 74.5 | 9.4 | 2.2 | 13.9 | (66 ± 1) |
| Tri 9 | 79.8 | 7.2 | 1.9 | 11.1 | (56.7 ± 0.7) |
| Tri 10 | 86.1 | 7.4 | 2.1 | 11.4 | (55.0 ± 0.7) |
| Tri 11 | 76.3 | 8.8 | 2.0 | 12.9 | (64.8 ± 0.4) |
| Tri 12 | 78.2 | 9.9 | 1.9 | 10.0 | (60.1 ± 0.8) |
| Tri 13 | 73.0 | 7.6 | 2.2 | 17.2 | (65.2 ± 0.5) |
| Tri 14 | 81.9 | 7.1 | 2.3 | 8.7 | (57.7 ± 0.3) |

TABLE 4
Tetra Salts

| | | | Additional | Additional | Band 3 |
|---|---|---|---|---|---|
| | AP soln. | CaCl₂ | Glycine | Water | HPLC |
| Sample ID | (% w/w) | (% w/w) | (% w/w) | (% w/w) | (%) |
| Tetra 1 | 83.1 | 12.5 | 3.0 | 1.4 | (75.5 ± 0.5) |
| Tetra 2 | 87.5 | 8.7 | 2.0 | 1.8 | (73.0 ± 0.5) |
| Tetra 3 | 75.0 | 9.8 | 2.7 | 12.5 | (75.2 ± 0.7) |
| Tetra 4 | 78.1 | 8.5 | 1.3 | 12.1 | (70.4 ± 0.4) |
| Tetra 5 | 80.4 | 8.1 | 1.4 | 10.1 | (68.8 ± 0.4) |
| Tetra 6 | 82.2 | 7.6 | 1.7 | 8.5 | (73.0 ± 0.5) |
| Tetra 7 | 84.1 | 7.0 | 2.1 | 6.8 | (76.0 ± 0.8) |
| Tetra 8 | 90.5 | 6.5 | 1.5 | 1.5 | (67.0 ± 0.2) |
| Tetra 9 | 77.6 | 10.2 | 1.8 | 10.4 | (72.8 ± 0.6) |
| Tetra 10 | 77.6 | 10.2 | 2.2 | 10.0 | (74.7 ± 0.3) |
| Tetra 11 | 72.8 | 10.5 | 1.7 | 15.0 | (73.8 ± 0.8) |
| Tetra 12 | 85.9 | 10.2 | 1.9 | 2.0 | (74.9 ± 0.5) |
| Tetra 13 | 88.1 | 9.0 | 2.3 | 0.6 | (74.2 ± 0.8) |
| Tetra 14 | 92.3 | 5.1 | 2.5 | 0.1 | (73.1 ± 0.8) |

TABLE 5
Penta Salts

| | | | Additional | Additional | Band 3 |
|---|---|---|---|---|---|
| | AP soln. | CaCl₂ | Glycine | Water | HPLC |
| Sample ID | (% w/w) | (% w/w) | (% w/w) | (% w/w) | (%) |
| Penta 1 | 76.2 | 9.5 | 6.4 | 7.9 | (83.7 ± 0.2) |
| Penta 2 | 80.0 | 8.4 | 5.6 | 6.0 | (80.1 ± 0.7) |
| Penta 3 | 78.4 | 9.6 | 6.6 | 5.4 | (84.9 ± 0.2) |
| Penta 4 | 85.1 | 6.7 | 4.6 | 3.6 | (74.7 ± 0.1) |
| Penta 5 | 72.3 | 10.5 | 6.7 | 10.5 | (81.9 ± 0.5) |
| Penta 6 | 74.2 | 11.0 | 7.2 | 7.6 | (81.5 ± 0.8) |
| Penta 7 | 90.3 | 4.4 | 4.4 | 0.9 | (56.7 ± 0.4) |
| Penta 8 | 82.6 | 8.3 | 6.3 | 2.8 | (81.3 ± 0.6) |
| Penta 9 | 79.6 | 6.5 | 5.8 | 8.1 | (80.2 ± 0.6) |
| Penta 10 | 87.5 | 5.7 | 4.4 | 2.4 | (60.4 ± 0.1) |
| Penta 11 | 81.4 | 8.6 | 6.1 | 3.9 | (80.0 ± 0.2) |
| Penta 12 | 82.9 | 9.5 | 5.1 | 2.5 | (78.2 ± 0.4) |
| Penta 13 | 77.2 | 10.7 | 7.0 | 5.1 | (81.6 ± 0.7) |
| Penta 14 | 77.9 | 10.0 | 6.5 | 5.6 | (82.4 ± 0.5) |

Reviewing Tables 2 to 5, it is clear that the highest Band 3 contents generally came from the penta salts, followed by the tetra salts, followed by the tri salts, followed by the octa salts.

In a further study, samples of each of the octa, penta, tetra and tri AP salt solutions (78.20%) were activated with the same weight percentages of calcium chloride dihydrate (7.32%), total glycine (4.45%) and additional water, using the method described above.

HPLC analysis of each of the solutions produced was performed by the method described above, producing the results indicated in Table 6.

TABLE 6

| | | | Total | Additional | | |
|---|---|---|---|---|---|---|
| | AP soln. | CaCl₂ | Glycine | Water | HPLC (%) | |
| Sample ID | (% w/w) | (% w/w) | (% w/w) | (% w/w) | Band 2 | Band 3 |
| Octa salt | 78.20 | 7.32 | 4.45 | To 100 | 31.25 +/− 3.0 | 53.25 +/− 1.2 |
| Penta salt | 78.20 | 7.32 | 4.45 | To 100 | 10.61 +/− 2.5 | 70.26 +/− 0.6 |
| Tetra salt | 78.20 | 7.32 | 4.45 | To 100 | 20.35 +/− 3.8 | 64.43 +/− 2.1 |
| Tri salt | 78.20 | 7.32 | 4.45 | To 100 | 19.49 +/− 2.6 | 63.67 +/− 1 3.7 |

The Band 3 to Band 2 ratio was by far the highest for the penta salt (6.6:1) and lowest for the Octa salt (1.7:1). The tetra salt and tri salt were 3.2:1 and 3.3:1, respectively.

In a further study, examples as indicated in Table 7 were prepared using the same activation method as described above. The levels of glycine and calcium chloride used in these examples were each expected to deliver maximum Band 3 content for each of the salts concerned, based upon computer modelling.

TABLE 7

| | AP | | Total | | | |
|---|---|---|---|---|---|---|
| | soln. | CaCl₂ | Glycine | Water | | |
| Al/Zr | (% | (% | (% | (% | Al:Zr | M:Cl |
| Salt | w/w) | w/w) | w/w) | w/w) | Ratio | Ratio |
| Octa | 75.2 | 8.5 | 5.3 | 14.2 | 8.41 | 0.99 |
| Penta | 73.6 | 10.4 | 7.0 | 9.1 | 6.70 | 1.16 |
| Tetra | 78.3 | 10.4 | 6.6 | 9.0 | 3.54 | 0.96 |
| Tri | 74.3 | 9.5 | 6.7 | 14.1 | 5.69 | 1.05 |

HPLC analysis of each of the solutions produced was performed by the method described above, producing the results indicated in Table 8. The average values and standard errors indicated are again based on 3 measurements. Also given are the molar ratios of aluminium to calcium and aluminium to glycine for the samples.

TABLE 8

| | Molar ratios | | | | |
|---|---|---|---|---|---|
| Al/Zr Salt | Al:Ca | Al:Glycine | Band 2/% | Band 3/% | Band 3:Band 2 |
| Octa | 3.77 | 3.05 | 30.0 ± 0.4 | 55.0 ± 0.6 | 1.83 |
| Penta | 3.33 | 2.53 | 11.1 ± 0.2 | 87.2 ± 0.3 | 7.85 |
| Tetra | 2.20 | 1.77 | 19.2 ± 0.2 | 71.3 ± 0.4 | 3.72 |
| Tri | 2.69 | 1.95 | 17.6 ± 0.3 | 69.4 ± 0.2 | 3.95 |

The results obtained were very similar to those reported in Table 6, with the Band 3 to Band 2 ratio being far highest for the penta salt and lowest for the octa salt.

Oil-in-water emulsion roll-on compositions were prepared using the components detailed in Table 9.

TABLE 9

| Ingredient | Function |
|---|---|
| Deionised Water | Balancing ingredient |
| Glycerol | Humectant |
| Steareth-20 | Emulsifier |
| Disodium EDTA | Preservative |
| Al/Zr solution | AP active |
| PPG-15 Stearyl Ether | Emollient |
| Steareth-2 | Emulsifier |
| Pentaerythrityl tetra-di-t-butyl | Oxidation Stabiliser |
| hydrocinnamate | |

The following procedure was used to prepare 3 kg samples of four different emulsion compositions. Deionised water (1120 g) was added to a 3 L beaker (1) charged with a magnetic stirrer bar. Glycerol (120 g), Steareth-20 (18 g), disodium EDTA (3 g) and Al/Zr solution (balanced with water to reach 12% (w/w of the final compositions) anhydrous Al/Zr active (excluding CaCl₂ and glycine) were added. The mixture was heated to 55° C. with stirring in a water bath until no solids remained. A separate 250 mL beaker (2) was charged with a magnetic stirrer bar, PPG-15 stearyl ether (120 g), steareth-2 (78 g) and pentaerythrityl tetra-di-t-butyl hydrocinnamate (1.5 g). The second beaker was heated to 90° C. using a water bath with stirring until no solids remained. The contents of (1) were then transferred to a further vessel (3). Whilst shearing at 10,000 rpm, the contents of (2) were added to the vessel (3) over 2 minutes. The resulting emulsion was left to cool to 45° C. before being sheared at 5000 rpm. pH measurements were made after 24 hours using a Mettler Toledo SevenCompact S220 with a Mettler Toledo Inlab® Expert Pro pH electrode (calibrated using buffers at pH 4, 7 and 10. All measurements were recorded at 25° C.

Further details of the four different oil-in-water emulsion compositions and their measured pH values are indicated in Table 10. The Al/Zr solutions used were prepared according to the method detailed above, adjusting CaCl₂ and glycine levels accordingly.

TABLE 10

| | Al/Zr salt | | Total | |
|---|---|---|---|---|
| Al/Zr | (anhydrous | CaCl₂•2H₂O | Glycine | |
| Salt | solids, % w/w) | (% w/w) | (% w/w) | pH |
| Octa | 12 | 4.23 | 2.64 | 3.44 +/− 0.03 |
| Penta | 12 | 4.66 | 3.85 | 3.74 +/− 0.02 |
| Tetra | 12 | 5.72 | 3.63 | 3.61 +/− 0.04 |
| Tri | 12 | 5.48 | 3.87 | 3.79 +/− 0.05 |

It can be seen that the emulsion composition with the Octa salt gave a significantly lower pH value, indicating a greater potential of irritation from this composition.

The oil-in-water emulsion compositions prepared as described above and detailed in Table 10 were stored at 45° C. in an oven and monitored over 12 weeks to assess rheological stability. Rheological stability was assessed in terms of the extent of (undesired) splitting of the compositions and the results are given in Table 11.

TABLE 11

| | Extent of splitting (% v/v) after . . . | | |
|---|---|---|---|
| Al/Zr Salt | 4 weeks | 8 weeks | 12 weeks |
| Octa | 1.92 | 9.62 | 1.54 |
| Penta | 0 | 4 | 4 |
| Tetra | 2 | 6 | 8 |
| Tri | 0 | 1.96 | 7.84 |

It is clear from these results that the composition with the penta salt had least extent of splitting after 12 weeks and hence the greater long term stability. The composition with the Octa salt had the greatest extent of splitting and hence the shortest long term stability.

Stability tests were also performed on the simple aqueous solutions of the Al/Zr solutions as indicated in Table 7 and the associated description. Each of the computer-optimised AP active solutions was stored in a 25° C. oven and monitored over 12 weeks. The samples were checked periodically and any changes noted. Full gelation was observed in the octa and tri samples, but not in tetra or penta samples, indicating superior rheological stability, in terms non-gelation, for these samples.

The aqueous-ethanol roll-on compositions indicated in Table 12 may be prepared from the relevant AP solution indicated in Table 7 using standard methods.

TABLE 12

| | Example | | | | | |
|---|---|---|---|---|---|---|
| Components | 1 | 2 | 3 | 4 | 5 | 6 |
| Ethanol | 23 | 20 | 25 | 54 | 50 | 60 |
| Penta Al/Zr | 17 | — | — | 26 | — | — |
| solution | | | | | | |
| Tetra Al/Zr | — | 16 | — | — | 20 | — |
| solution | | | | | | |
| Tri Al/Zr | — | — | 18 | — | — | 22 |
| solution | | | | | | |
| Glycerine | 3 | 2.5 | 1.5 | — | — | — |
| PEG-40 | 1.5 | 2 | 3 | — | — | — |
| hydrogenated | | | | | | |
| castor oil | | | | | | |
| Hydroxyethyl | 1 | 1 | 1 | 1 | 1 | 1 |
| cellulose | | | | | | |
| Fragrance | 1 | 0.5 | 0.7 | 07 | 1 | 0.5 |
| Additional | To 100 | To 100 | To 100 | To 100 | To 100 | To 100 |
| water | | | | | | |

1. An aqueous composition comprising:
an aluminium-zirconium-glycine complex which is an aluminium-zirconium penta-chlorohydrex-glycine complex, and
a calcium salt comprising a solubility in water of 10 g/L or greater;
wherein the molar ratio of aluminium to calcium is from 1.3 to 60 and the molar ratio of aluminium to glycine is from 1.5 to 25.

2. An aqueous composition according to claim 1, wherein the composition comprises ethanol.

3. An aqueous composition according to claim 1, having a pH of 3.5 to 4.1.

4. An aqueous composition according to claim 1, wherein the molar ratio of aluminium to calcium is from 1.8 to 20.

5. An aqueous composition according to claim 1, wherein the molar ratio of aluminium to glycine is from 1.75 to 1:10.

6. An aqueous composition according to claim 1, wherein the calcium salt is calcium chloride.

7. An aqueous composition according to claim 1, comprising a fragrance or an emollient oil.

8. An aqueous composition according to claim 1, wherein the aluminium-zirconium-glycine complex is heat activated by the calcium salt.

9. An aqueous composition according to claim 8, wherein the aluminium-zirconium-glycine complex is heat activated by the water-soluble calcium salt for at least 30 mins, at a temperature of at least 50° C.

10. A non-therapeutic method of reducing perspiration from the surface of the human body, comprising the topical application of a composition according to claim 1.

11. A method of manufacture of an aqueous composition according to claim 1, comprising heating together the aluminium-zirconium-glycine complex and the water-soluble calcium salt in aqueous solution at a molar ratio of aluminium to calcium of from 1.3 to 60 for at least 30 mins, at a temperature of at least 50° C., prior to cooling the mixture to ambient temperature.

12. A method of manufacture of an aqueous composition according to claim 11, wherein the resulting aqueous composition is subsequently mixed with fragrance and/or a cosmetically acceptable carrier.
